(19) United States
(12) Patent Application Publication
(10) Pub. No.: US 2026/0294765 A1
(43) Pub. Date: Oct. 1, 2026

(54) LAMELLAR WASH COMPOSITION NOT RELIANT ON PALM KERNEL OIL DERIVED STRUCTURANT

(71) Applicant: Conopco Inc., d/b/a UNILEVER, Hoboken, NJ (US)

(72) Inventors: Douglas John HIBAN, Newtown, CT (US); Teanoosh MOADDEL, Watertown, CT (US); Massimiliano TRENTINI, Old Saybrook, CT (US); Tirucherai Varahan VASUDEVAN, Bethany, CT (US)

(73) Assignee: Conopco Inc., d/b/a UNILEVER, Hoboken, NJ (US)

(21) Appl. No.: 19/479,616

(22) PCT Filed: May 3, 2024
(86) PCT No.: PCT/EP2024/062239

§ 371 Date: Oct. 28, 2025

(30) Foreign Application Priority Data
May 4, 2023 (EP) 23171494.0

(51) Int. Cl. A61K 8/46; A61K 8/41; A61K 8/44; A61Q 19/10

(52) U.S. Cl. CPC A61K 8/466; A61K 8/41; A61K 8/44; A61Q 19/10

(57) ABSTRACT

The present invention is directed to a lamellar wash composition having a surfactant system with an anionic that is at least 40% by weight isethionate, and an amphoteric, zwitterionic or both. The composition is stable and has no or reduced amounts of surfactant with an amidoal-kyl bridge and no or reduced amounts of structurant derived from palm kernel oil. The composi-tion surprisingly provides excellent sensory and lathering characteristics and remains in lamellar phase.

### FIELD OF THE INVENTION

The present invention is directed to a lamellar wash composition having a reduced amount or no palm kernel oil derived lamellar structurant. More particularly, the wash composition comprises anionic surfactant that is at least 40% by weight isethionate, and an amphoteric surfactant, zwitterionic surfactant or both wherein at least 60% by weight of the amphoteric and zwitterionic surfactants used are each independently free of an amidoalkyl bridge. The wash composition of the present invention is surprisingly stable and suitable to be formulated substantially free of at least one of sulfate, paraben, hydantoin, isothiazolinone, dioxane and silicone oil. The composition also, and unexpectedly, provides excellent lather and sensory characteristics, with total surfactant in the wash composition being at least 80% by weight amphoteric surfactant, zwitterionic surfactant or both, and anionic surfactant comprising at least 40% by weight isethionate.

### BACKGROUND OF THE INVENTION

Wash compositions are typically employed to cleanse skin and to reduce shine associated with sebum produced in specialized epithelial cells known as sebocytes. They are also used to minimize bacteria on the hands and face such that washing is viewed as the most effective way to prevent the spread of germs and bacteria. In fact, experts believe that periodic washing throughout the day can reduce the number of consumers catching colds by about 50%. In addition to skin, wash compositions are conventionally used on the scalp and hair and are also used to clean objects within the home, like counters, windows, and cloths.

Consumers prefer compositions that are mild, especially on skin and hair, and they also demand compositions that are more favorable to the environment. Particularly, use of less ingredients to make compositions is desirable to simplify composition production, resulting in a more ecofriendly manufacturing process.

It is of increasing interest to make wash compositions that are enjoyed by consumers and better for the planet. The present invention, therefore, is directed to a lamellar wash composition having a reduced amount or no palm kernel oil derived lamellar structurant. More particularly, the wash composition comprises anionic surfactant that is at least 40% by weight isethionate, and an amphoteric surfactant, zwitterionic surfactant or both wherein at least 60% by weight of the amphoteric and zwitterionic surfactants used are each independently free of (do not comprise) an amidoalkyl bridge. The wash composition of the present invention is surprisingly stable and suitable to be formulated substantially free of at least one of sulfate, paraben, hydantoin, isothiazolinone, dioxane and silicone oil. The composition also, and unexpectedly, provides excellent lather and sensory characteristics, with total surfactant in the wash composition being at least 80% by weight amphoteric surfactant, zwitterionic surfactant or both, and anionic surfactant comprising at least 40% by weight isethionate.

### Additional Information

Efforts have been disclosed for making wash compositions. In World Application No. WO/03017968A2, lamellar compositions and aerosol barrier dispensing systems are described.

Other efforts have been described for making detergent compositions. In U.S. Published Patent Application No. US 2016/0186095, detergent compositions with amine oxide, alkylbenzene sulfonate, alkyl ether sulfate, and fatty alcohol ethoxylate are described.

Still other efforts have been described for making wash compositions. In European Patent No. EP 3971275 B1, a liquid hand dishwashing composition having at least 50% by weight anionic surfactant which is an alkyl sulfate and cosurfactant including a betaine or betaine and amine oxide, and polypropylene glycol is described.

Even other efforts have been described for making wash compositions. In U.S. Pat. No. 5,807,816, a light duty cleansing composition with alkyl sulfate, alkyl ether sulfate, a betaine, amine oxide or mixture thereof and alkylpolyglucoside are described.

None of the additional information describes a lamellar wash composition with a reduced amount or no palm kernel oil derived lamellar structurant as claimed herein.

### SUMMARY OF THE INVENTION

In a first aspect, the present invention is directed to a lamellar wash composition comprising:
a) an anionic surfactant comprising at least 40% by weight isethionate;
b) amphoteric surfactant, zwitterionic surfactant or both;
c) 0 to 5% by weight nonionic surfactant; and
d) water,
wherein the composition:
i) has a viscosity of at least 30,000 mPa-s;
ii) a pH of at least 5.65;
iii) comprises no more than 3% by weight of a sulfate comprising surfactant;
iv) at least 60% by weight of the amphoteric surfactant and zwitterionic surfactant are each independently free of an amidoalkyl bridge;
v) the amphoteric surfactant is at least 80% by weight amine oxide; and
vi) at least 60% by weight of the zwitterionic surfactant in the wash composition has a hydrophilic portion that is —N⁺—(R)₂—(C(R¹)₂)ₓ—COO— or —N⁺—(R)₂—(C(R²)₂)_{y}—SO₃— where each R is independently H or a C₁₋₃ alkyl; each R¹ is independently H or a C₁₋₃ alkyl; x is an integer from 1 to 3; each R² is independently H, OH or a C₁₋₄ alkyl and y is an integer from 1-6.

In a second aspect, the present invention is directed to a method for washing or treating a surface, like skin, with the lamellar wash composition of the first aspect of the invention.

In a third aspect, the invention is directed to the use of an anionic surfactant comprising at least 40% by weight isethionate in combination with an amphoteric surfactant, zwitterionic surfactant or both to make a lamellar wash composition that is substantially free of or free of palm kernel oil derived lamellar structurant, the amphoteric surfactant and zwitterionic surfactants each independently being at least 60% free of an amidoalkyl bridge.

All other aspects of the present invention will more readily become apparent from the description and examples which follow.

Skin, as used herein, is meant to include skin on the arms (including underarms), face, back, feet, neck, chest, hands, legs, buttocks and scalp (including hair). Stable means able to maintain a viscosity of at least 30,000 mPa-s, and preferably, at least 35,000 mPa-s, and most preferably, from 35,000 to 345,000 mPa-s (or from 40,000 to 240,000 mPa-s or from 43,000 to 138,000 mPa-s or from 44,000 to 75,000 mPa-s) for at least one month, and preferably, at least 2 months after being made and stored at 45° C. where viscosity is taken at 25° C. with a Discovery HR-2 Rheometer using sand blasted plates having a 1000-micron gap and a shear rate of 0.23 s⁻¹.

Palm kernel oil derived lamellar structurant means a component having a fatty portion with at least 75 to 100% (or 80 to 100% or 90 to 100% or 95 to 100% or 100%) by weight of a hydrophobic chain that is a C₈-C₁₄ saturated hydrocarbon from palm kernel oil and including components like lauric and myristic acid or their alcohol or amide derivatives. Lamellar, as used herein, means having layers of surfactant arrangements where polar head groups align with water to shield fatty acid acyl chains from the water such that over 85% (or 90 to 100% or 100%) of the total resulting surfactants in the composition are arranged as micelle plates or layers creating a translucent or opaque composition, where the surfactant arrangement is induced by the surfactants themselves and as selected in the invention, creating a creamy (e.g., velvety) wash composition. Amidoalkyl bridge as used herein means a group represented as —(C═O)—N(R)—Z— where R is as previously defined and Z is a linear, branched or cyclic alkyl but often is linear and a C₁₋₈ alkyl, and more often a C₁₋₄ or C₁₋₃ alkyl, and most often, a propyl group whereby such group links the surfactant polar head and hydrophobic tail. Again, at least 60% by weight of the amphoteric surfactant and zwitterionic surfactant used are each independently free of the amidoalkyl bridge, and preferably, at least 65%, and most preferably, at least 70% (or 77.5 to 96% or 78 to 86.5% or 77 to 94% or 79.5 to 100% or 95 to 100% or 97 to 100% or 100%) are each independently free of such bridge.

In the present invention, it has been unexpectedly discovered that use of anionic surfactant comprising at least 40% by weight isethionate, and amphoteric surfactant and/or zwitterionic surfactant with at least 60% by weight of the same being free of the amidoalkyl bridge can be used to induce or make a stable lamellar wash composition with reduced or even no palm kernel oil derived structurant. Thus, the surfactant system used in the present invention surprisingly results in a lamellar wash composition having excellent sensory and lather characteristics and such surfactant system mimics wash composition structuring that is typically seen with traditional palm kernel oil based structurant. In an embodiment of the invention, less than 2.25%, and preferably, less than 2%, and most preferably, less than 1.5% (or from 0.0 to 1.2% or from 0.01 to 0.75% or from 0.01 to 0.4%, or less than 0.25% or 0.0%) by weight of the lamellar wash composition comprises structurant derived from palm kernel oil (like lauric and myristic acid or their alcohol or amide derivatives). The lamellar wash composition of this invention is one suitable to be wiped or washed off, and preferably, washed off with water. Such a composition can be a home care cleaning composition (e.g., for tabletops, glass, toilets dishes, upholstery, or laundry) but is preferably a shampoo, make-up wash, facial wash, hand wash, or personal care wash like a liquid body wash. In an embodiment of the invention, when the lamellar wash composition is used for hand applications, the composition will preferably have a viscosity that is not higher than 170,000 mPa-s, and is preferably, from 30,000 to 125,000 mPa-s, and most preferably, from 30,000 to 115,000 mPa-s.

The lamellar wash composition of this invention may, optionally, comprise medicinal or therapeutic agents, but preferably, is a wash which is for cosmetic uses and non-therapeutic formulated to remove dirt, oil or the like from surfaces including skin and hair. In a preferred embodiment, the lamellar wash composition is a personal wash composition, and therefore, a liquid body wash for use while showering or bathing. The lamellar wash composition of the present invention may optionally comprise skin benefit ingredients added thereto such as emollients, vitamins and/or derivatives thereof, resorcinols, retinoic acid precursors, colorants, moisturizers, sunscreens, antibacterial agents, mixtures thereof or the like. Such skin benefit ingredients (or agents) can be water and/or oil soluble. If used, oil soluble skin benefit agents typically make up to 2.0% by weight of the lamellar wash composition whereby water-soluble skin benefit agents, when optionally used, may make up to 15% by weight of the wash composition of the present invention.

The lamellar wash composition will typically have a pH from 5.65 to 8 or 5.7 to 7.75 or from 5.75 to 7.5 or from 5.8 to 7.25 or from 6 to 7 or from 6.15 to 6.85.

As used herein, “substantially free of” as it relates to sulfate means less than 3.0%, and preferably, less than 2.5%, and most preferably, less than 2% or less than 1%, and even more preferably, less than 0.75% (or less than 0.5%) by weight of the lamellar wash composition. In an embodiment of the invention, “substantially free of sulfate includes a wash composition with 0.0% (i.e., none) by weight sulfate comprising ingredient, including a sulfate-based surfactant. Regarding the non-sulfate-based ingredients (i.e., ingredients, like a hydantoin, that are not, for example, anionic sulfate comprising surfactants), substantially free of means less than 0.7% by weight, and preferably, less than 0.4%, and most preferably, less than 0.3%, (or less than 0.2% or less than 0.1% or less than 0.05%) by weight of the wash composition. As to the non-sulfate-based ingredients, substantially free of also includes 0.0% (i.e., none) by weight of the ingredient in the wash composition. With respect to all ingredients that the wash composition of the present invention may be substantially free of, it is within the scope of the invention to include from 0.001 to 0.05% by weight of such ingredient. In yet another preferred embodiment, the wash composition of the present invention comprises less than 35 ppm, and preferably, less than 25 ppm, and most preferably, less than 15 ppm dioxane or less than 2 ppm or less than 1 ppm dioxane or 0.0 ppm dioxane based on total dioxane in the wash composition. In another embodiment, the wash composition of this invention may comprise from 0.00001 to 0.00005% by weight dioxane, like 1,4-dioxane.

The term comprising is meant to encompass the terms consisting essentially of and consisting of. For the avoidance of doubt, and for illustration, the wash composition of this invention comprising surfactant and active is meant to include a composition consisting essentially of the same and a composition consisting of the same. All ranges defined herein are meant to include all ranges subsumed therein. Except in the operating comparative examples, or where otherwise explicitly indicated, all numbers in this description indicating amounts or ratios of materials or conditions and/or physical properties of materials and/or use are to be understood as modified by the word “about”. The disclosure, as found herein, is to be considered to cover all embodiments as found in the claims as being multiply dependent upon each other irrespective of the fact that claims may be found without multiple dependency or redundancy.

### DETAILED DESCRIPTION OF THE INVENTION

As to the amphoteric surfactant suitable for use, the same is at least 80%, and preferably, at least 85%, and most preferably, at least 90% (or 90 to 100% or 94 to 100% or 94 to 98% or 100%) by weight amine oxide whereby at least 60% by weight of the amine oxide does not have (i.e., is free of) the defined amidoalkyl bridge, and has the formula: R³—N⁺ (R⁴)₂—O— where R³ is a C₈₋₂₀ alkyl, and preferably, a C₁₀₋₁₈ alkyl, and most preferably, a C₁₂₋₁₈ alkyl (or C₁₂₋₁₆ alkyl) and R⁴ is H or a C₁₋₆ alkyl or C₁₋₄ alkyl or C₁₋₃ alkyl or C₁₋₂ alkyl or —CH₃. In an embodiment of the invention, the amine oxide is cocoyl amine oxide, lauramine oxide, myristamine oxide, palmitamine oxide, stearamine oxide, oleamine oxide or mixtures thereof.

Amine oxide suitable for optional use, in addition to the amine oxides described above, include those having an amidoalkyl bridge like cocamidopropyl amine oxide, lauryl amidopropyl amine oxide, myristyl amidopropyl amine oxide, palmityl amidopropyl amine oxide, stearyl amidopropyl amine oxide, oleamidopropyl amine oxide or a mixture thereof. Again, when amine oxide is used, at least 60% by weight of the amine oxide does not have an amidoalkyl bridge.

Additional amphoteric surfactants suitable for use (i.e., non-amine oxides) include include imdazolines, sodium acyl amphoacetates, sodium acyl amphopropionates, disodium acyl amphodiacetates and disodium acyl amphodipropionates where the acyl (i.e., alkanoyl group) can comprise a C₇-C₁₈ alkyl portion. Illustrative examples of the amphoteric surfactants suitable for use include sodium lauroamphoacetate, sodium cocoamphoacetate, sodium lauroamphoacetate, sodium cocoamphoacetate, cocamphodipropionate, or mixtures thereof.

When used, amphoteric surfactant makes up from 1.5 to 15% or from 3.5 to 15% by weight of the lamellar wash composition, and preferably, from 4 to 13%, and most preferably, from 4.5 to 12% or 4.75 to 11% or from 4.8 to 10.5% by weight of the wash composition.

As to the zwitterionic surfactant suitable for use, at least 60% by weight of the surfactant used does not have (i.e., is free of) the defined amidoalkyl bridge and includes those represented as R³—N⁺—(R)₂—(C(R¹)₂)ₓ—COO— or —N⁺—(R)₂—(C(R²)₂)_{y}—SO₃— or a mixture thereof where x, y, R, R¹, R² and R³ are as previously defined. Illustrative examples of the zwitterionic surfactant suitable for use include lauryl betaine, myristyl betaine, palmityl betaine, stearyl betaine, oleyl betaine, lauryl hydroxy sultaine, myristyl hydroxy sultaine, palmityl hydroxy sultaine, stearyl hydroxy sultaine, oleyl hydroxy sultaine or a mixture thereof. In an embodiment of the invention, the zwitterionic surfactant is lauryl betaine, myristyl betaine, palmityl betaine or a mixture thereof, or lauryl betaine, myristyl betaine, or a mixture thereof, or lauryl betaine.

When used, zwitterionic surfactant free of amidoalkyl group makes up from 1.5 to 9.5, and preferably, from 2.5 to 8.5, and most preferably, from 3.5 to 7.5 or from 4.5 to 7% by weight of the lamellar wash composition. In an embodiment of the invention, the total amount of amphoteric and zwitterionic surfactant used is from 5 to 20%, and preferably, from 6 to 18%, and most preferably, from 7 to 16% or from 7 to 13% or from 7.5 to 12% or from 7.3 to 11% by weight of the lamellar wash composition.

In another embodiment of the invention, amphoteric surfactant and zwitterionic surfactant are both included at a weight ratio of 1:4 to 4:1, or from 1:3 to 3:1 or from 1:2 to 2:1, or from 1:1.5 to 1.5:1 or from 1:1.2 to 1.2:1 or from 1:1. In still another embodiment, lauryl amine oxide is used with cocoyl betaine, lauryl betaine, lauryl sultaine or both, and preferably, lauryl amine oxide is used with cocoyl betaine, lauryl betaine. In even another embodiment, only amphoteric surfactant is used or only zwitterionic surfactant is used like lauryl amine oxide or lauryl betaine.

Anionic surfactants in the present invention are at least 40% by weight isethionate (i.e., acyl isethionate). Other anionic surfactants suitable for optional use in the wash composition of the present invention include aliphatic sulfonates, such as a primary alkane (e.g., C₈-C₂₂) sulfonate, primary alkane (e.g., C₈-C₂₂) disulfonate, C₈-C₂₂ alkene sulfonates, C₈-C₂₂ hydroxyalkane sulfonates or alkyl glyceryl ether sulfonates (AGS); or aromatic sulfonates such as alkyl benzene sulfonate. The anionic may also include alkyl sulfates (e.g., C₁₂-C₁₈ alkyl sulfates) or alkyl ether sulfates (including alkyl glyceryl ether sulfates). Among the alkyl ether sulfates are those having the formula:

R⁴O(CH₂CH₂O)ₙSO₃M

wherein R⁴ is an alkyl or alkenyl having 8 to 18 carbons, preferably 12 to 18 carbons, n has an average value of at least 1.0, preferably less than 5, and most preferably 1 to 4, and M is a solubilizing cation such as sodium, potassium, ammonium or substituted ammonium.

The anionic may also optionally include alkyl sulfosuccinates (including mono- and dialkyl, e.g., C₆-C₂₂ sulfosuccinates); alkyl and acyl taurates (often methyl taurates), alkyl and acyl sarcosinates, sulfoacetates, C₈-C₂₂ alkyl phosphates and phosphonates, alkyl phosphate esters and alkoxyl alkyl phosphate esters, acyl lactates, C₈-C₂₂ monoalkyl succinates and maleates, sulphoacetates, alkyl glucosides and acyl isethionates, or the like.

Sulfosuccinates may be monoalkyl sulfosuccinates having the formula:

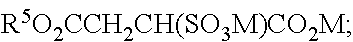

and amide-MEA sulfosuccinates of the formula:

R⁵CONHCH₂CH₂O₂CCH₂CH(SO₃M)CO₂M wherein R⁵ ranges from C₈-C₂₂ alkyl.

Sarcosinates are generally indicated by the formula:

R⁶CON(CH₃)CH₂CO₂M, wherein R⁶ ranges from C₈-C₂₀ alkyl.

Taurates are generally identified by formula:

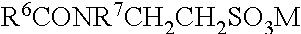

wherein R⁶ is as previously described and R⁷ is a C₁-C₄ alkyl.

M is a solubilizing cation as previously described.

Isethionates that may be used and make up at least 40% by weight of the anionic surfactant used in the lamellar wash composition of the present invention include C₈-C₁₈ acyl isethionates (including those which have a substituted head group such as a C₁₋₄ alkyl substitution, preferably methyl substitution). These surfactant esters are prepared by a reaction between alkali metal isethionate with mixed aliphatic fatty acids having from 6 to 18 carbon atoms and an iodine value of less than 20. Often at least 75% of the mixed fatty acids have from 12 to 18 carbon atoms and up to 25% have from 6 to 10 carbon atoms.

The acyl isethionate suitable for use may be an alkoxylated isethionate such as is described in Ilardi et al., U.S. Pat. No. 5,393,466, entitled “Fatty Acid Esters of Polyalkoxylated isethonic acid; issued Feb. 28, 1995; hereby incorporated by reference. This compound has the general formula:

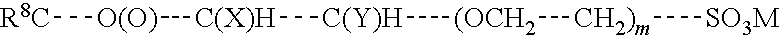

wherein R⁸ is an alkyl group having 8 to 18 carbons, m is an integer from 1 to 4, X and Y are each independently hydrogen or an alkyl group having 1 to 4 carbons and M is a solubilizing cation as previously described.

Additional anionic surfactants suitable for optional use in the lamellar wash compositions of the present invention are acyl glycinates and acyl glutamates or mixtures thereof. These include anionics like sodium lauroyl glycinate, sodium cocoyl glycinate, potassium lauroyl glycinate, potassium cocoyl glycinate, sodium lauroyl glutamate, sodium cocoyl glutamate, potassium lauroyl glutamate, potassium cocoyl glutamate or mixtures thereof.

Illustrative isethionates include sodium lauroyl isethionate, sodium methyl lauroyl isethionate and sodium cocoyl isethionate, and illustrative taurates include sodium methyl lauroyl taurate, sodium methyl cocoyl taurate or mixtures of any of the same. Such anionic surfactants are commercially available. Sodium cocoyl isethionate, sodium methyl lauroyl taurate, sodium lauroyl glycinate, sodium lauroyl glutamate, sodium methyl lauroyl isethionate or mixtures thereof are the preferred anionics suitable for use. In an embodiment of the invention, the anionic surfactant used, in addition to an isethionate, is at least one of sodium lauroyl glycinate, and/or sodium methyl lauroyl taurate. In a preferred embodiment, the anionic surfactant used in the wash composition of this invention is sodium lauroyl isethionate with sodium lauroyl glycinate, sodium lauroyl taurate, sodium methyl lauroyl taurate or a mixture thereof. In yet another preferred embodiment, the anionic surfactant is an isethionate with taurate, and preferably, sodium lauroyl isethionate and sodium methyl lauroyl taurate. In even another embodiment of the invention, the anionic surfactant is all (i.e., 100%) by weight isethionate like sodium lauroyl isethionate, sodium methyl lauroyl isethionate, sodium cocoyl isethionate or a mixture thereof. Anionic surfactant that is 95 to 100% or 100% by weight isethionate is often preferred.

In yet another embodiment of the invention, anionic surfactants suitable for use can optionally include alpha-olefin sulfonates (Na or K C₁₄-C₁₈, preferably, C₁₄-C₁₆). Even others include ethylene oxide sulfonates (EOS) or mixtures of alpha-olefin sulfonates and ethylene oxide sulfonates in a weight ratio of 2:8 or 8:2, or 3:7 to 7:3, or 4:6 to 6:4.

Anionic surfactant typically makes up at least from 1.5 to 18% (or 2 to 18%), and preferably, from 2.5 to 16%, and most preferably, from 2.7 to 15% or from 2.8 to 14% or from 2.85 to 13% by weight of the lamellar wash composition. In another embodiment of the invention, anionic surfactant makes up from 2.9 to 7.6 of from 2.9 to 7.2% by weight of the lamellar wash composition.

As to the anionic surfactant, again, at least 40% by weight of the total anionic surfactant in the lamellar wash composition is isethionate, and more preferably, at least 45% and most preferably, from 45 to 100%, or from 45 to 90% or from 47 to 85% or from 47 to 78% or from 48 to 58% by weight of the total anionic surfactant in the lamellar wash composition is isethionate. In another embodiment, the total anionic surfactant in the lamellar wash composition is 100% by weight isethionate or from 40 to 60% or from 45 to 55% by weight isethionate when the anionic surfactant used is a mixture of isethionate and taurate.

The surfactant system used in the present invention can be used with traditional palm kernel oil derived lamellar structurants or no palm kernel oil derived structurants as described herein (i.e., less than 2.25% by weight of the wash composition comprising palm kernel oil structurant). Structurant (or structuring agent) like a C₆-C₁₄ acid, alcohol and/or amide (i.e., hydroxylated or amide derivatives of the acid thereof) can optionally be included and such structurants will, again, make up less than 2.25%, and preferably, less than 2%, and most preferably, less than 1.5% or from 0.0 to 1.2% (or from 0.01 to 0.7% or 0.01 to 0.4% or less than 0.25% or 0.0%) by weight of the total weight of the lamellar wash composition.

As to the palm kernel oil derived structurant suitable for optional use in the invention, such traditional palm kernel oil derived structurants include caproic, caprylic, capric, lauric and/or myristic acid, and any alcohol or amide derivatives of the same.

Additional structurants suitable for optional use (alone or in combination with the palm kernel oil structurants described herein) include those generally classified as lactylates such as a C₁₆-C₂₀ lactylates. In an embodiment of the invention, the preferred lactylates are C₁₆-C₁₈ lactylates, and more preferably, C₁₆-C₁₈ lactylates like palmitoyl-1-lactylate, stearoyl-1-lactylate or mixtures thereof. Polylactyls (typically numbering from 2 to three lactyl groups) are also suitable for use, like palmitoyl-2-lactylate, stearoyl-2-lactylate or mixtures thereof. In another embodiment of the invention, the lactylate is a mixture of palmitoyl-1-lactylate and palmitoyl-2-lactylate, or stearoyl-1-lactylate stearoyl-2-lactylate or a mixture thereof. In still another embodiment of the invention, the lactylate is sodium stearoyl-1-lactylate and sodium stearoyl-2-lactylate where sodium stearoyl-2-lactylate makes up from 30 to 80% or 40 to 70% or 55 to 65% by weight of the lactylate mixture. If used the lactylates will make up less than 2% by weight of the lamellar wash composition, or from 0.01 to 1.65% or from 0.02 to 1.3% or from 0.03 to 0.8% or 0.0% by weight of the lamellar wash composition. Counter ions in addition to sodium are also suitable for use such as ammonium and potassium cations.

Fatty acid sources for the structurant and amphoteric and zwitterionic surfactants suitable to be used in this invention can, for example, include vegetable, soy, nut, fruit (e.g., mango, papaya, red berries, coconut) and/or palm oil. In an embodiment, of the invention, less than 95%, and preferably, less than 90%, and most preferably, less than 85% by weight of the fatty acid sources for the structurant, amphoteric and/or zwitterionic surfactants used includes hydrophobic chain derived from palm kernel oil or palm kernel oil, or both.

In even another embodiment, the structurant, zwitterionic and amphoteric surfactant used in the present invention can optionally comprise from 1 to 15%, or 1 to 12%, or 2 to 11% or 2 to 6% or 0.00005 1.5% or 0.0001 to 1.0% or 0.001 to 0.5% by weight carbon recovered from carbon capture (e.g., purple carbon) based on total weight of carbon in sturcturant, amphoteric and/or zwitterionic surfactant.

Thickener like polymeric carbohydrate thickener is suitable for use in the present invention, the same can include nonmodified starch granules, in general, like potato starch, waxy maize starch as well as simple corn starch, i.e., a starch that gelatinizes at around 75° C. Pure-Gel® starches from Grain Processing Corporation are chemically modified corn starch granules having a gelatinization temperature at about 53° C. and are often preferred for use. Modified and/or nonmodified starch granules with gelatinization temperatures from 30° to 85° C., and preferably, from 30° to 80° C., and most preferably, from 35 to 75° C. are suitable for inclusion as the polymeric carbohydrate thickening agent in the lamellar wash compositions of the invention. In general, and notwithstanding the starch selected for use, it is typically preferred that the granules of the polymeric carbohydrate selected, upon use in the final lamellar wash composition, swell at least 200%, and preferably at least 400%, and more preferably, at least 600%, and most preferably, at least 800% by volume in the lamellar wash composition to form swollen starch gel particles with a size in the range of 2 to 300 micrometers, or preferably, from 3 to 275 microns, and most preferably, from 4 to 245 microns. Examples of the often preferred Pure-Gel® starches suitable for use are Pure Gel B990, Pure Gel B992, Pure Gel B980 and Pure Dent starches made commercially available from Grain Processing Corporation. Additional polymeric carbohydrates suitable for use (i.e., starch granules) are National™ 1545, Amioca corn starch, Clearjel, National 1333, Colflo 67, Novation 1600, Novation 2700 or Purity 420 made available from Ingredion. Chemically modified starch granules are also suitable for use. Starch granules modified with nonionic hydrophilic groups such as hydroxyethyl or hydroxypropyl and/or ionic groups such as phosphate, carboxylate, sulfate, sulfonate and dialkyl/trialkyl amino or quaternary ammonium ions are often selected for use.

Even other polymeric carbohydrates suitable for use are water soluble starches like Ultra-Sperse®, tapioca and waxy maize starch, and National 1215 pregelatinized unmodified corn starch or mixtures thereof. Still others include Structure® ZEA, a hydroxypropyl modified corn starch, or Structure® XL, a cross-linked pregelatinized hydroxypropyl starch phosphate or mixtures thereof, whereby the same are also available commercially from Nouryon.

Other useful thickeners that can be used include carbohydrate gums such as cellulose gum, microcrystalline cellulose, cellulose gel, hydroxyethyl cellulose, hydroxypropyl cellulose, sodium carboxymethylcellulose, hydroxymethyl or carboxymethyl cellulose, methyl cellulose, ethyl cellulose, guar gum, gum karaya, gum tragacanth, gum Arabic, gum acacia, gum agar, xanthan gum and mixtures thereof; modified and nonmodified starch granules often with gelatinization temperatures between 30 to 85° C.

Additional thickeners that also may be used include Versathix™ (PEG-150 Pentaerythrityl Tetrastearate (and) PPG-2 Hydroxyethyl cocamide and water), Aristoflex AVC (ammonium acryloyldimethyltaurate/VP copolymer), Carbomer such as Carbopol 980 (crosslinked polyacrylic acid), Carbopol® Ultrez 10 and 20 (hydrophobically modified crosslinked polyacrylic acid); alkaline soluble emulsion polymers such as Aculyn 28, Acuyln 22 or Carbopol Aqua SF1; cationic polymer such as modified polysaccharides including cationic guar available from Aventis under the trade name Jaguar C13S, Jaguar C14S, Jaguar C17, or Jaguar C16; cationic modified cellulose such as UCARE Polymer JR 30 or JR 40 from Amerchol; N-Hance 3000, N-Hance 3196, N-Hance GPX 215 or N-Hance GPX 196 from Ashland, Inc.; synthetic cationic polymer such as MerQuat 100, MerQuat 280, Merquat 281 and Merquat 550 by Nalco; cationic starches, e.g., StaLok® 100, 200, 300 and 400 made by Galactasol 800 series by Henkel, Inc.; Quadrosoft Um-200; Polyquaternium-67 and Polyquaternium-24.

Thickener used in the lamellar wash composition of the present invention preferably comprises at least 75% (or at least 85%, or from 90 to 100% or 100%) by weight polymeric carbohydrate. In another embodiment the polymeric carbohydrate is at least 50% by weight or 55 to 100% or 65 to 95% or 75 to 90% or 80 to 90% or 90 to 98% or at least 95% or 100% by weight starch where starch is defined to mean polymer, plant derived and comprising D-Glucose and 1,4 and/or 1,6 alpha-glycosidic bonds or linkages and preferably both.

In still another embodiment, when thickener is used that is not a polymeric carbohydrate, the thickener selected typically makes up less than 25%, and preferably, less than 15%, and most preferably, from 0.0 to 6% (or 0.001 to 3% or 0.0001 to 2%) by weight of the total weight of thickener used in the lamellar wash composition.

The total amount of thickener used often makes up from 2 to 12%, and preferably, from 2.25 to 10%, and most preferably, from 2.5 to 9.5% (or 2.75 to 9% or 2.85 to 8.25% or 2.85 to 6.2%) by weight of the wash composition.

In yet another embodiment of the invention, less than 1%, and preferably, less than 0.5%, and most preferably, less than 0.25% or 0.0% by weight of the thickener used in the lamellar wash composition is acrylate based.

As to oils suitable for optional use in the lamellar wash compositions of this invention, the same include any oils suitable for topical application via a wash composition, and preferably, an oil suitable to contact skin that is natural and sustainable. Illustrative examples of such oils include silicone oils and/or mineral oil, but preferably those that are naturally sourced and sustainable like, arachis oil, castor oil, coconut oil, corn oil, cotton seed oil, olive oil, rapeseed oil, safflower seed oil, sesame seed oil, soybean oil, avocado oil, macadamia nut oil, argan oil, pomegranate oil, argan Moroccan oil, blueberry oil, raspberry oil, walnut oil, pecan oil, peanut oil, bayberry oil, mango seed oil, jojoba oil, hydrolyzed jojoba oil, mixtures thereof or the like. If a silicone oil is optionally used, the same can include, for example, PEG-3 Dimethicone, PEG-8 Dimethicone, PEG-9 Dimethicone, PEG-10 Dimethicone, PEG-11 Methyl ether dimethicone, PEG-12 Dimethicone, PEG-14 Dimethicone, PEG-17 Dimethicone, PEG-32 Dimethicone mixtures thereof or the like.

In an embodiment of the invention, the lamellar wash composition of the present invention comprises from 0.5 to 5% by oil, and preferably, from 1.0 to 4.5%, and most preferably, from 1.5 to 4.0% (or from 1.75 to 3.5% or from 2.0 to 3.0%) by weight oil based on total weight of oil in the wash composition. In another embodiment, the oil used is at least 70%, or 80% or 90 to 100% or 100% by weight soybean oil when used. In even another embodiment, the lamellar was composition of the present invention comprises less than 0.2% by weight oil or no (0.0%) by weight oil. In still another embodiment, when oil is used, less than 0.5% or 0.0% by weight of the total weight of the oil used is a silicone oil.

Zwitterionic surfactants having the amidoalkyl bridge as described herein can optionally be used, making up no more than 40% by weight of the zwitterionic surfactant used in the invention. Such surfactants include at least one acid group and the acid group may be a carboxylic or a sulphonic acid group. Such zwitterionic surfactants often include quaternary nitrogen, and therefore, can be quaternary amino acids. They also should generally include an alkyl or alkenyl group of 7 to 18 carbon atoms generally comply with an overall structural formula:

R⁹—[—C(O)—NH(CH₂)_{q}—]ᵣ—N⁺—(R¹⁰—)(R¹¹) A-B where R⁹ is alkyl or alkenyl of 7 to 18 carbon atoms; R¹⁰ and R¹¹ are each independently alkyl, hydroxyalkyl or carboxyalkyl of 1 to 3 carbon atoms; q is 2 to 4; r is 0 to 1; A is alkylene of 1 to 3 carbon atoms optionally substituted with hydroxyl, and B is —CO₂— or —SO₃—.

In an optional embodiment of the invention, therefore, zwitterionic surfactants that are amido betaines of the formula: R⁹—CONH(CH₂)ₜ—N⁺—(R¹⁰)(R¹¹)CH₂CO₂ where t is 2 or 3 can optionally be used. In an embodiment of the invention, less than 3.5% or less than 3% or less than 2.5% or less than 2% or less than 1.5% or from 0.01 to 1.25% or 0.0% of betaine and/or sultaine having an amidoalkyl bridge is used in the lamellar wash composition of the invention.

In both formulae R⁹, R¹⁰ and R¹¹ are as defined previously. R⁹ may, in particular, be a mixture of C₁₂ and C₁₄ alkyl groups derived from coconut oil so that at least half, preferably at least three quarters of the groups R⁹ have 10 to 14 carbon atoms. R¹⁰ and R¹¹ are preferably methyl.

A further possibility is that the zwitterionic surfactant is a sulphobetaine of the formula:

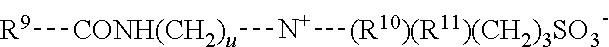

where u is 2 or 3, or variants of these in which —(CH₂)₃SO₃ is replaced by CH₂C(OH)(H)CH₂SO₃⁻.

In these formulae, R⁹, R¹⁰ and R¹¹ are as previously defined.

Illustrative examples of the zwitterionic surfactants with amidoalkyl group suitable for use include betaines like cocodimethyl carboxymethyl betaine, and cocamidopropyl betaine and laurylamidopropyl betaine. An additional zwitterionic surfactant suitable for use includes cocamidopropyl sultaine. Such surfactants are made commercially available from suppliers like Stepan Company, and it is within the scope of the invention to employ mixtures of the aforementioned surfactants. In a preferred embodiment and when used, the zwitterionic surfactant having that has an amidoalkyl group is cocamidopropyl betaine.

In an embodiment of the invention, less than 3.5% or less than 3% or less than 2.5% or less than 2% or less than 1.5% or from 0.01 to 1.25% or 0.0% of betaine and/or sultaine having an amidoalkyl bridge is used in the lamellar wash composition of the invention.

In another embodiment, when amphoteric surfactant that is not an amine oxide is used, such surfactant should not exceed the amount of amine oxide used. Preferably, and when present, amphoteric surfactant that is not an amine oxide is used at an amount that is less than 50% or less than 40% or from 0.5 to 25% by weight of the total weight of amine oxide used in the lamellar wash composition. In even another embodiment, the amount of amphoteric surfactant used that is not an amine oxide is from 0.5 to 10% by weight of the total weight of amine oxide in the lamellar wash composition. In yet another embodiment, no amphoteric surfactant that is not an amine oxide is used.

In a further embodiment, when zwitterionic surfactant that comprises an amidoalkyl bridge is used, such surfactant should not exceed the amount used of zwitterionic surfactant that does not comprise an amidoalkyl bridge. Preferably, and when present, zwitterionic surfactant that does comprise an amidoalkyl bridge is used, the same is present at an amount that is less than 50% or less than 40% or from 0.5 to 25% by weight of the total weight of zwitterionic surfactant used that does not comprise an amidoalkyl bridge. In even another embodiment, the amount of zwitterionic surfactant that does comprise an amidoalkyl bridge is from 0.5 to 10% by weight of the total weight of zwitterionic surfactant that does not comprise an amidoalkyl bridge in the lamellar wash composition. In yet another embodiment, no zwitterionic surfactant that does comprise an amidoalkyl bridge is used.

Nonionic surfactants may optionally be used in the wash composition of the present invention. When used, nonionic surfactants are typically used at levels as low as 0.25, 1, 1.5 or 2% by weight and at levels as high as 3, 4, 5 or 6% by weight of the lamellar wash composition, when used. The nonionics which may be used include the reaction products of compounds having a hydrophobic group and a reactive hydrogen atom, for example aliphatic alcohols, acids, amides or alkylphenols with alkylene oxides, especially ethylene oxide either alone or with propylene oxide. Specific nonionic surfactant compounds are alkyl (C₆-C₂₂) phenols ethylene oxide condensates, the condensation products of aliphatic (C₈-C₁₈) primary or secondary linear or branched alcohols with ethylene oxide, and products made by condensation of ethylene oxide with the reaction products of propylene oxide and ethylenediamine. Other nonionic surfactants include long chain tertiary amine oxides, long chain tertiary phosphine oxides, dialkyl sulphoxides, and the like.

In an embodiment of the invention, nonionic surfactants optionally used can include fatty acid/alcohol ethoxylates having the following structures a) HOCH₂(CH₂)ₛ(CH₂CH₂O)ᵥH or b) HOOC(CH₂)_{c}(CH₂CH₂O)_{d}H; where s and v are each independently an integer up to 18; and c and d are each independently an integer from 1 or greater. In an embodiment of the invention, s and v are each independently 6 to 18; c and d are each independently 1 to 30. Other options for nonionic surfactants include those having the formula HOOC(CH₂)ᵢ—CH═CH—(CH₂)ₖ(CH₂CH₂O)_{z}H, where i, k are each independently 5 to 15; and z is 5 to 50. In another embodiment of the invention, i and k are each independently 6 to 12; and z is 15 to 35.

The nonionic used may also include a sugar amide, such as a polysaccharide amide. Specifically, the surfactant may be one of the lactobionamides described in U.S. Pat. No. 5,389,279 to Au et al., entitled “Compositions Comprising Nonionic Glycolipid Surfactants issued Feb. 14, 1995; which is hereby incorporated by reference or it may be one of the sugar amides described in U.S. Pat. No. 5,009,814 to Kelkenberg, titled “Use of N-Poly Hydroxyalkyl Fatty Acid Amides as Thickening Agents for Liquid Aqueous Surfactant Systems” issued Apr. 23, 1991; hereby incorporated into the subject application by reference.

In an embodiment of the invention, the nonionic surfactant used is an alkyl polyglucoside represented as:

R¹²—O—(S)ₚ, where R¹² is a C₆ to C₂₀ or C₈ to C₁₈ or C₁₀ to C₁₆ linear or branched alkyl and S is a saccharide group of 5 or 6 carbons such as glucose, xylose, lactose, fructose and/or mannose, and preferably, is glucose, and p represents the degree of polymerization and may have a value of from 1 to 12 or from 1 to 10, and most preferably, from 1.2 to about 1.6 or from 1.3 to 1.5.

In an embodiment of the invention, the nonionic surfactant used is cocamide monoethanolamine (cocamide MEA) and nonionic makes up from 0.2 to 1% or from 0.25 to 0.85% or from 0.3 to 0.5% by weight of the wash composition.

In still another embodiment of the invention, cationic surfactants may optionally be used in the wash composition of the present invention.

One class of optional cationic surfactants includes heterocyclic ammonium salts such as cetyl or stearyl pyridinium chloride, alkyl amidoethyl pyrrylinodium methyl sulfate, and lapyrium chloride.

Tetra alkyl ammonium salts are another useful class of cationic surfactants suitable for optional use. Examples include cetyl or stearyl trimethyl ammonium chloride or bromide; hydrogenated palm or tallow trimethylammonium halides; behenyl trimethyl ammonium halides or methyl sulfates; decyl isononyl dimethyl ammonium halides; ditallow (or distearyl)dimethyl ammonium halides, and behenyl dimethyl ammonium chloride.

Still other types of cationic surfactants that may be used are the various ethoxylated quaternary amines and ester quats. Examples include PEG-5 stearyl ammonium lactate (e.g., Genamin KSL manufactured by Clariant), PEG-2 coco ammonium chloride, PEG-15 hydrogenated tallow ammonium chloride, PEG 15 stearyl ammonium chloride, dipalmitoyl ethyl methyl ammonium chloride, dipalmitoyl hydroxyethyl methyl sulfate, and strearyl amidopropyl dimethylamine lactate.

Even other useful cationic surfactants suitable for optional use include quaternized hydrolysates of silk, wheat, and keratin proteins, and it is within the scope of the invention to use mixtures of the aforementioned cationic surfactants.

If used, cationic surfactants will make up no more than 1.0% by weight of the lamellar wash composition. When present, they typically make up from 0.01 to 0.7%, and more typically, from 0.1 to 0.5% by weight of the wash composition.

In an embodiment of this invention, the wash composition of this invention will be substantially free of polymeric quaternary ammonium compounds (including salts of the same). In another embodiment, the wash composition will comprise less than 0.1% by weight polymeric quaternary ammonium compounds. In yet another embodiment, the wash composition comprises less than 0.01% by weight polymeric quaternary ammonium compounds. In even another embodiment, the wash composition is free of polymeric quaternary ammonium compounds (i.e., 0.0%).

Inorganic salt is an optional but often desired ingredient to aid in wash composition thickening. Typical salts may be used like NaCl, KCl, MgCl₂, CaCl₂), mixtures thereof or the like. Typically, the inorganic salt makes up from 0 to 5%, and preferably, from 0.1 to 4%, and most preferably, from 0.25 to 2.5% (or from 0.5 to 2%) by weight of the composition.

Polyols (i.e., humectants) are suitable for optional use in the lamellar wash compositions of the present invention whereby the same are limited only to the extent that they are suitable for use in a topical wash composition and water soluble. Illustrative and nonlimiting examples of the polyols suitable for use in the present invention include sorbitol, glycerol, mannitol, xylitol, maltitol or mixtures thereof. In an embodiment of the invention, the polyol used is at least 50% by weight glycerol, based on total weight of the polyol used in the wash composition. In another embodiment of the invention, the polyol used is all glycerol (100% by weight). Polyol, when used, will typically make up from 1 to 40% by weight of the wash composition, and preferably, from 1.5 to 25% by weight of the wash composition, and most preferably, from 2 to 15% (or from 2.5 to 10%) by weight of the lamellar wash composition of the present invention.

Water typically makes up from 30 to 90%, and preferably, from 40 to 90%, and most preferably, from 50 to 85% by weight of the lamellar wash composition or even from 68 to 78% by weight of the composition.

Adjusters suitable to modify/buffer the pH may be used. Such pH adjusters include triethylamine, NaOH, KOH, H₂SO₄, HCl, C₆H₈O₇ (i.e., citric acid) or mixtures thereof. The pH adjusters are added at amounts to yield the desired final pH. The pH values may be assessed with commercial instrumentation such as a pH meter made commercially available from Thermo Scientific®.

Optional water-soluble skin benefit agents suitable for use in the wash composition of this invention are limited only to the extent that they are capable of being topically applied, and suitable to dissolve in the wash composition at the desired pH.

Illustrative examples of the benefit agents suitable to include in the water portion of the wash composition are acids, like amino acids, such as arginine, valine or histidine. Additional water-soluble benefit agents suitable for use include vitamin B2, niacinamide (vitamin B3), vitamin B5 (Panthenol), vitamin B6, vitamin C, mixtures thereof or the like. Water soluble derivatives of such vitamins may also be employed. For instance, vitamin C derivatives such as ascorbyl tetraisopalmitate, magnesium ascorbyl phosphate and ascorbyl glycoside may be used alone or in combination with each other. Other water-soluble benefit agents suitable for use include 4-ethyl resorcinol, extracts like sage, aloe vera, green tea, grapeseed, thyme, chamomile, yarrow, cucumber, liquorice, rosemary extract or mixtures thereof. Water soluble sunscreens like ensulizole may also be used. Total amount of optional water-soluble benefit agents (including mixtures) when present in the invention may range from 0.0 to 15%, preferably from 0.001 to 10%, and most preferably, from 0.01 to 8% (or from 1 to 6%) by weight of the wash composition.

It is also within the scope of the present invention to optionally include oil soluble benefit agents (i.e., non-water-soluble agents defined as less than 1.0 g of such benefit agent (solute) can be dissolved in 100 mL of 25° C. water (solvent) at atmospheric pressure). The only limitations with respect to such oil soluble benefit agents are that the same are suitable to provide a benefit when topically applied, especially to skin.

Illustrative examples of the types of oil soluble benefit agents that may optionally be used in the compositions of this invention include components like stearic acid, vitamins like Vitamin A, D, E and K (and their oil soluble derivatives), sunscreens like ethylhexylmethoxycinnamate, bis-ethyl hexyloxyphenol methoxyphenol triazine, 2-ethylhexyl-2-cyano-3,3-diphenyl-2-propanoic acid, drometrizole trisiloxane, 3,3,5-trimethyl cyclohexyl 2-hydroxybenzoate, 2-ethylhexyl-2 hydroxybenzoate or mixtures thereof.

Other well known sunscreens suitable for use include Mexoryl 400 (methoxypropylamino cyclohexenylidene ethoxyethylcyanoacetate), Mexoryl XL (drometrizole trisiloxane), Mexoryl SX (terephthalylidene dicamphor sulfonic acid) or mixtures thereof.

Other optional oil soluble benefit agents suitable for use include resorcinols like thiamidol (isobutylene thiazolyl resorcinol), 4-hexyl resorcinol, 4-phenylethyl resorcinol, 4-cyclopentyl resorcinol, 4-cyclohexyl resorcinol 4-isopropyl resorcinol or a mixture thereof. Also, 5-substituted resorcinols like 4-cyclohexyl-5-methylbenzene-1,3-diol, 4-isopropyl-5-methylbenzene-1,3-diol, mixtures thereof or the like may be used. The 5-substituted resorcinols, and their synthesis are described in commonly assigned U.S. Published Patent Application No. 2016/0000669A1.

Even other oil soluble actives suitable for use include omega-3 fatty acids, omega-6 fatty acids, climbazole, farnesol, ursolic acid, myristic acid, geranyl geraniol, oleyl betaine, cocoyl hydroxyethyl imidazoline, hexanoyl sphingosine, 12-hydroxystearic acid, petroselinic acid, conjugated linoleic acid, terpineol, thymol mixtures thereof or the like.

In an embodiment of the invention, the optional oil soluble benefit agent used is a retinoic acid precursor. In one embodiment of the invention, the retinoic acid precursor is retinol, retinal, retinyl propionate, retinyl palmitate, retinyl acetate or a mixture thereof. Retinyl propionate, retinyl palmitate and mixtures thereof are typically preferred.

Still another retinoic acid precursor suitable for use is hydroxyanasatil retinoate made commercially available under the name Retextra® as supplied by Molecular Design International. The same may be used in a mixture with the oil soluble actives described herein.

When optional oil soluble active is used in the composition of the invention, such active typically makes up from 0.0 to 2%, and preferably, from 0.001 to 1.5%, and most preferably, from 0.05 to 1% (or from 0.06 to 0.85%) by weight of the lamellar wash composition.

Conventional preservatives can desirably be incorporated into the wash composition to protect against the growth of potentially harmful microorganisms. Cosmetic chemists are familiar with appropriate preservatives and routinely choose them to satisfy the preservative challenge test and to provide product stability. Suitable traditional preservatives for use may include hydantoin derivatives methyl paraben, propyl paraben and propionate salts. Preservatives suitable for use may include iodopropynyl butyl carbamate, phenoxyethanol, hydroxyacetophenone, ethylhexylglycerine, hexylene glycol, imidazolidinyl urea, sodium dehydroacetate, dimethyl-dimethyl (DMDM) hydantoin, benzyl alcohol, sodium benzoate or mixtures thereof. Other preservatives suitable for use include sodium dehydroacetate, chlorophenesin, isothiazolinones (CIT/MIT) and decylene glycol. The preservatives should be selected having regard for the use of the composition and possible incompatibilities between the preservatives and other ingredients in the emulsion. Preservatives are preferably employed in amounts ranging from 0.01% to 2.0% by weight of the total weight of the wash composition, including all ranges subsumed therein. Also preferred is a preservative system with hydroxyacetophenone alone or in a mixture with other preservatives. Use of vicinal diols like 1,2-octane diol are also suitable for use with the preservatives described herein and typically at amounts from 0.2 to 1.5%, and preferably, from 0.3 to 1.25%, and most preferably, from 0.3 to 1% by weight of the wash composition.

In an embodiment of the invention, the wash composition is substantially free of hydantoins and parabens and isothiazolinones as described therein.

Fragrances, fixatives, chelators (like EDTA and/or sodium gluconate) and exfoliants may optionally be included in the wash composition of the present invention. Each of these substances may range from about 0.03 to about 5%, preferably between 0.1 and 3% (or from 0.2 to 1.2%) by weight of the total weight of the wash composition. To the extent the exfoliants are used, those selected should be of small enough particle size so that they do not impede the performance of any packaging used to dispense the compositions of this invention.

Conventional emulsifiers having an HLB of greater than 8 may optionally be used. Illustrative examples include Tween, 40, 60, 80, polysorbate 20 and mixtures thereof. Typically, emulsifiers for water continuous systems make up from 0.03 to 1.5% by weight of the wash composition.

As to the lamellar wash composition of the present invention, the same typically comprises from 5 to 20%, and preferably, from 6 to 18%, and most preferably, from 7 to 17% (or from 8 to 14%) by weight total surfactant. In an embodiment of the invention, the wash composition comprises from 7 to 13.2% by weight surfactant.

In an embodiment of the invention, well-known sunscreens suitable for use include Mexoryl 400 (methoxypropylamino cyclohexenylidene ethoxyethylcyanoacetate), Mexoryl XL (drometrizole trisiloxane), Mexoryl SX (terephthalylidene dicamphor sulfonic acid) or mixtures thereof.

In another embodiment of the invention, the lamellar wash composition of the present invention comprises palmitic acid, glycerol, and peroxisome proliferator-activated receptor ligands (“PPARs”) like 12-hydroxystearic acid, petroselinic acid, conjugated linoleic acid, stearic acid and mixtures of such PPARs. When used, the weight percents of the ingredients (i.e., skin matrix building ingredients) in the lamellar wash compositions are 0.1 to 6%, and 1 to 10%, and 0.1 to 3.5%, respectively.

In even another embodiment of the invention, at least one of 1,2-hexanediol, 1,2-nonanediol tetrahexyldecyl ascorbate, honokiol, hyaluronic acid, salicylic acid, lemongrass oil, climbazole, benzalkonium chloride, hinokitiol, piroctone olamine, linalool, linseed oil, flaxseed oil, benzalkonium chloride, cetrimonium chloride, benzalkonium chloride, benzethonium chloride, anisic acid, levulinic acid, chloride, cetylpyridinium chloride, pyruvic acid, flaxseed oil hemp seed oil and/or magnolia bark extract is used in the lamellar wash composition of the invention. If used, the same either alone or in a mixture make up from 0.001 to 5%, or from 0.01 to 4% or from 0.01 to 3.5% or from 0.02 to 2.7% or from 0.02 to 1% by weight of the lamellar wash composition.

When making lamellar wash composition of the present invention, the desired ingredients may be mixed with conventional apparatus under moderate shear and atmospheric conditions, with temperature being from 30 to 85° C. whereby shear continues until a homogeneous product is recovered.

The packaging for the wash composition typically is not limited as long as composition can be dispensed. In an embodiment on the invention, the wash composition is sold in a pouch, bottle, jar, tube or canister. The packaging preferably is refillable, biodegradable and/or is prepared from recycled materials including post-consumer resins.

The Examples are provided to facilitate an understanding of the invention. They are not intended to limit the scope of the claims.

All Samples in the Examples were prepared by mixing the ingredients with moderate shear and with temperature being about 80° C. and pressure being atmospheric. Viscosity (millipascal second) was measured with a Discovery HR-2 Rheometer using sand blasted plates having a 1000-micron gap and a shear rate of 0.23 s⁻¹.

### Example I

In this Example I, seven Samples of body wash compositions were prepared as shown in Table I. The wash compositions were made with the surfactants described. Hydrogenated soybean oil was present at 3% by weight of the composition and non-hydrogenated soybean oil was present at 2% by weight of the composition. Polyquaternium 67 was used at 0.8% by weight and guar (Jaguar C14S) was used at 0.2% by weight of the composition. Samples 1 to 6 also had 4.5% by weight starch, Puregel B990 at 4.5% by weight.

Caprylyl Glycol was used at 0.3% by weight and stearic acid was present at 0.25% by weight of the composition. Humectant was present at 1% by weight as was 0.05% chelating agent. Fragrances were included at about 1.15% by weight percent along with pH adjusters to achieve a pH between 6.5 and 7.5. The resulting wash compositions had about 70% by weight water. Sodium Benzoate was used to preserve the compositions (0.5% by weight) and the viscosities of the compositions consistent with the invention were about 80,000 mPa-s.

As can be observed from the data presented in Table I, when surfactants are selected according to the invention, the resulting wash compositions are surprisingly stable in lamellar phase and have excellent sensory and lather characteristics even when formulated with less than 2.25% by weight of palm kernel oil derived structurant. Consistent with the invention, when at least 40% by weight of the total weight of anionic surfactant in the composition is an isethionate, no crystallization is observed and the compositions are lamellar wash compositions with no palm kernel oil derived ingredient necessary.

TABLE I

| | Sample | Sample | Sample | Sample | Sample | Sample | Sample |
|---|---|---|---|---|---|---|---|
| Ingredients | 1 | 2 | 3 | 4 | 5 | 6 | 7 |
| Sodium Lauroyl | 10 | 6.6 | 6.5 | 3 | 5 | 6.5 | 9.5 |
| Isethionate | | | | | | | |
| Sodium Methyl | 0 | 6.6 | 0 | 0 | 0 | 0 | 0 |
| Lauroyl | | | | | | | |
| Taurate | | | | | | | |
| Lauramine Oxide | 10 | 6.6 | 6.5 | 7 | 5 | 0 | 9.5 |
| Coco Betaine | 0 | 0 | 0 | 0 | 0 | 6.5 | 0 |
| Microstrucure | Lamellar | Lamellar | Lamellar | Lamellar | Lamellar | Lamellar | Lamellar |

* Confirmed via visual inspection of the Samples

### Example II

In this Example II, the wash compositions, Samples 8 to 11, were prepared in a manner similar to the one described in Example 1 but with ingredients as shown in Table II. Hydrogenated soybean oil was present at 3% by weight of the composition and non-hydrogenated soybean oil was present at 2% by weight of the composition. Caprylyl Glycol was present at 0.3% by weight and stearic acid was present at 0.25% by weight of the composition. Humectant was used at 1% as was 0.05% by weight a chelating agent. Fragrances were added at 1.15% by weight of the wash compositions and pH adjusters were used to achieve a pH between 6.5 and 7.5. The compositions had about 70% by weight water. Sodium Benzoate was used to preserve the compositions (0.5% by weight) and each composition had 4.5% by weight starch, Puregel B990. The viscosities for the compositions not consistent with the invention were about 20,000 mPa-s.

As can be observed from the data presented in Table II, when surfactants are not selected according to the invention, the resulting wash compositions are not stable (i.e., show visible crystallization), are isotropic and not lamellar, exhibit a scratchy sensory feel and lather poorly when compared to traditional lamellar compositions prepared with palm kernel oil derived structurants and compared to the compositions described in Example I.

TABLE II

| Ingredients | Sample 8 | Sample 9 | Sample 10 | Sample 11 |
|---|---|---|---|---|
| Sodium Lauroyl | 6.5 | 0 | 0 | 0 |
| Isethionate | | | | |
| Sodium Methyl Lauroyl | 0 | 8.5 | 0 | 0 |
| Taurate | | | | |
| Sodium Lauroyl | 0 | — | 6.5 | 8.5 |
| Glycinate | | | | |
| Lauramine Oxide | 0 | 0 | 0 | 8.5 |
| Coco Betaine | 0 | 8.5 | 6.5 | 0 |
| Lauric Acid | 0 | 0 | 0 | 0 |
| Cocamidopropyl | 6.5 | 0 | 0 | 0 |
| hydroxysultaine | | | | |
| Microstrucure | Isotropic/ | Isotropic/ | Isotropic/ | Isotropic/ |
| | Crystaline | Crystaline | Crystaline | Crystaline |

*Confirmed via visual inspection of the Sample

1. A lamellar wash composition comprising:
a) an anionic surfactant comprising at least 40% by weight isethionate, based on total anionic surfactant used in the lamellar wash composition;
b) amphoteric surfactant, zwitterionic surfactant or both;
c) 0 to 5% by weight nonionic surfactant, based on the weight of the composition; and
d) water,
wherein the composition:
i) has a viscosity of at least 30,000 mPa-s, wherein viscosity is taken at 25° C. with a Discovery HR-2 Rheometer using sand blasted plates having a 1000-micron gap and a shear rate of 0.23 s⁻¹;
ii) a pH of at least 5.65;
iii) comprises no more than 3% by weight of a sulfate comprising surfactant;
iv) at least 60% by weight of the amphoteric surfactant and zwitterionic surfactant are each independently free of an amidoalkyl bridge, being a group represented as —(C═O)—N(R)—Z— where R is H or a C₁₋₃ alkyl, and Z is a linear, branched or cyclic alkyl;
v) the amphoteric surfactant is at least 80% by weight amine oxide; and/or
vi) at least 60% by weight of the zwitterionic surfactant in the wash composition has a hydrophilic portion that is —N⁺—(R)₂—(C(R¹)₂)ₓ—COO— or —N⁺—(R)₂—(C(R²)₂)_{y}—SO₃— where each R is independently H or a C₁₋₃ alkyl; each R¹ is independently H or a C₁₋₃ alkyl; x is an integer from 1 to 3; each R² is independently H, OH or a C₁₋₄ alkyl and y is an integer from 1-6;
vii) comprises less than 0.27% by weight oil.

2. The lamellar wash composition according to claim 1 wherein from 45 to 90% by weight of the anionic surfactant is isethionate.

3. The lamellar wash composition according to claim 1 wherein the isethionate is sodium lauroyl isethionate, sodium methyl lauroyl isethionate, sodium cocoyl isethionate or a mixture thereof.

4. The lamellar wash composition according to claim 1 wherein the composition further comprises a taurate, glycinate, glutamate or a mixture thereof.

5. The lamellar wash composition according to claim 1 wherein the zwitterionic surfactant is present and comprises lauryl betaine, myristyl betaine, palmityl betaine, stearyl betaine, oleyl betaine, lauryl hydroxy sultaine, myristyl hydroxy sultaine, palmityl hydroxy sultaine, stearyl hydroxy sultaine, oleyl hydroxy sultaine or a mixture thereof.

6. The lamellar wash composition according to claim 1 wherein the amphoteric surfactant is present and comprises cocoyl amine oxide, lauramine oxide, myristamine oxide, palmitamine oxide, stearamine oxide, oleamine oxide or mixtures thereof.

7. The lamellar wash composition according to claim 1 wherein the pH of the composition is 5.65 to 8.

8. The lamellar wash composition according to claim 1 wherein the viscosity of the composition is 35,000 to 345,000 mPa-s.

9. The lamellar wash composition according to claim 1 wherein the composition has less than 0.4% by weight of at least one of a sulfate-based surfactant, paraben, hydantoin, isothiazolinone, dioxane and silicone oil.

10. The lamellar wash composition according to claim 1 wherein the composition is free of palm kernel oil derived lamellar structurant, and further wherein the composition comprises less than 0.2 wt % of oil.

11. The lamellar wash composition according to claim 1 wherein the composition further comprises niacinamide, 12-hydroxystearic acid, vitamin A, vitamin D, vitamin E, vitamin K or a mixture thereof.

12. The lamellar wash composition according to claim 1 wherein the composition further comprises thiamidol, 4-hexyl resorcinol, sunscreen or a mixture thereof.

13. The lamellar wash composition according to claim 1 wherein the composition further comprises 1,2-hexanediol, 1,2-nonanediol, honokiol, hyaluronic acid, salicylic acid, lemongrass oil, climbazole, benzalkonium chloride, hinokitiol, piroctone olamine, linalool, linseed oil, flaxseed oil, benzalkonium chloride, magnolia bark or a mixture thereof.
